(19) United States
(12) Patent Application Publication
(10) Pub. No.: US 2024/0024634 A1
(43) Pub. Date: Jan. 25, 2024

(54) Integrated Catheter with Needle-Free Connector

(71) Applicant: Becton, Dickinson and Company, Franklin Lakes, NJ (US)

(72) Inventors: Erica E. Neumann, Sandy, UT (US); Evan Barbour VandenBrink, San Mateo, CA (US); Ralph L. Sonderegger, Farmington, UT (US); John M. Lackey, West Valley City, UT (US); Benjamin P. Hopwood, West Valley City, UT (US); Jonathan Karl Burkholz, Salt Lake City, UT (US)

(21) Appl. No.: 18/478,047

(22) Filed: Sep. 29, 2023

Related U.S. Application Data
(63) Continuation of application No. 17/898,617, filed on Aug. 30, 2022.
(60) Provisional application No. 63/239,180, filed on Aug. 31, 2021.

(51) Int. Cl. A61M 25/06; A61M 25/00

(52) U.S. Cl. CPC A61M 25/0606; A61M 25/0097; A61M 2039/1077

(57) ABSTRACT

An integrated intravenous catheter includes a catheter adapter having a catheter and an inlet, with the catheter configured to be inserted into a patient's vasculature, a needle-free connector comprising a first port, a second port positioned opposite the first port, and a side port positioned the first port and the second port, with the second port including a valve member, intermediate tubing extending between the inlet of the catheter adapter and the first port of the needle-free connector, and extension tubing extending from the side port of the needle-free connector.

### CROSS-REFERENCE TO RELATED APPLICATION

The present application is a continuation of U.S. patent application Ser. No. 17/898,617 filed on Aug. 30, 2022, entitled “Integrated Catheter with Needle-Free Connector”, which claims priority to U.S. Provisional Application Ser. No. 63/239,180, entitled “Integrated Catheter with Needle-Free Connector”, filed Aug. 31, 2021, the entire disclosures of which are hereby incorporated by reference in their entirety.

### BACKGROUND OF THE INVENTION

### Field of the Invention

The present disclosure relates to an integrated catheter with a needle-free connector.

### Description of Related Art

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of catheter is an over-the-needle peripheral intravenous (“IV”) catheter (“PIVC”). The over-the-needle catheter may be mounted over an introducer needle having a sharp distal tip. The catheter and the introducer needle may be assembled so that the distal tip of the introducer needle extends beyond the distal tip of the catheter with the bevel of the needle facing up away from a skin surface of the patient. The catheter and introducer needle are generally inserted at a shallow angle through the skin into the vasculature of the patient. In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

Blood withdrawal using a peripheral IV catheter may be difficult for several reasons, particularly when an indwelling time of the catheter is more than one day. For example, when the catheter is left inserted in the patient for a prolonged period of time, the catheter or vein may be more susceptible to narrowing, collapse, kinking, blockage by debris (e.g., fibrin or platelet clots), and adhering of a tip of the catheter to the vasculature. Due to this, catheters may often be used for acquiring a blood sample at a time of catheter placement but are much less frequently used for acquiring a blood sample during the catheter dwell period.

Accordingly, blood draw devices have been developed to collect blood samples through an existing PIVC. Blood draw devices attach to the PIVC and include a flexible flow tube that is advanced through the PIVC, beyond the catheter tip, and into a vessel to collect a blood sample. After blood collection, the blood draw device is removed from the PIVC and discarded. One example of a blood draw device is shown and described in U.S. Pat. No. 11,090,461, which is hereby incorporated by reference in its entirety.

### SUMMARY OF THE INVENTION

In one aspect or embodiment, an integrated intravenous catheter includes a catheter adapter having a catheter and an inlet, with the catheter configured to be inserted into a patient's vasculature, a needle-free connector including a first port, a second port positioned opposite the first port, and a side port positioned between the first port and the second port, with the second port having a valve member, intermediate tubing extending between the inlet of the catheter adapter and the first port of the needle-free connector, and extension tubing extending from the side port of the needle-free connector.

The needle-free connector may include a body defining a flow path extending between the first port and the second port, where the side port is offset from a center of the flow path. The intermediate tubing may have a length that is shorter than a length of the extension tubing. The intermediate tubing may have a length configured to allow a probe of a blood draw device extend a predetermined length beyond a tip of the catheter. The intermediate tubing may have a length of 6-8 mm. The intermediate tubing may have a maximum length of 15 mm. The integrated catheter may include a medical connector positioned at an end of the extension tubing. The intermediate tubing may be fixedly connected to the inlet of the catheter adapter and the first port of the needle-free connector. The first port of the needle-free connector may be connected to the intermediate tubing via a line connector.

The needle-free connector may include a body defining a longitudinal axis extending between the first port and the second port, where the side port extends from the body at an angle of 30-150 degrees relative to the longitudinal axis of the body. The body of the needle-free connector may include a first portion and a second portion connected to the first portion via a luer connector.

At least a portion of the needle-free connector may be transparent. The needle-free connector may include an anti-reflux valve. The needle-free connector may include internal structure configured to create a vortex when fluid enters the needle-free connector via the side port.

The needle-free connector may include a stabilization member configured to contact a skin surface of a patient during use of the integrated intravenous catheter. The stabilization member may be formed integrally with the needle-free connector. The stabilization member may be separately connected to the needle-free connector.

In one aspect or embodiment, a method of flushing the integrated catheter of any of the above aspects or embodiments, includes: disconnecting a blood draw device from the second port of the needle-free connector; connecting a reservoir comprising a flushing fluid to the needle-free connector; and flushing the first port, the second port, and the side port of the needle-free connector and the catheter of the catheter adapter with the flushing fluid via only one of the ports of the needle-free connector.

The reservoir may be a flush syringe or an intravenous fluid container. The reservoir may be connected to the side port of the needle-free connector.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a perspective view of an integrated catheter according to one aspect or embodiment of the present application;

FIG. 2 is a perspective view of an integrated catheter according to one aspect or embodiment of the present application;

FIG. 3 is an enlarged perspective view of the integrated catheter of FIG. 2, showing a needle-free connector;

FIG. 4 is a perspective view of the integrated catheter of FIG. 2, showing a blood draw device connected to a needle-free connector with a flow tube of the blood draw device extended;

FIG. 5 is a perspective view of a needle-free connector according to one aspect or embodiment of the present application.

FIG. 6 is an exploded view of the needle-free connector of FIG. 5;

FIG. 7 is a perspective view of an integrated catheter according to one aspect or embodiment of the present application;

FIG. 8 is a front view of a needle-free connector according to one aspect or embodiment of the present application;

FIG. 9 is a cross-sectional view of the needle-free connector of FIG. 9;

FIG. 10 is a side view of the needle-free connector of FIG. 9;

FIG. 11 is a front view of a needle-free connector according to one aspect or embodiment of the present application;

FIG. 12 is a cross-sectional view of the needle-free connector of FIG. 11; and

FIG. 13 is a perspective view of an integrated catheter according to one aspect or embodiment of the present application.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION OF THE INVENTION

Spatial or directional terms, such as “left”, “right”, “inner”, “outer”, “above”, “below”, and the like, are not to be considered as limiting as the invention can assume various alternative orientations.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary aspects of the invention.

Unless otherwise indicated, all ranges or ratios disclosed herein are to be understood to encompass the beginning and ending values and any and all subranges or subratios subsumed therein. For example, a stated range or ratio of “1 to 10” should be considered to include any and all subranges or subratios between (and inclusive of) the minimum value of 1 and the maximum value of 10; that is, all subranges or subratios beginning with a minimum value of 1 or more and ending with a maximum value of 10 or less.

The terms “first”, “second”, and the like are not intended to refer to any particular order or chronology, but refer to different conditions, properties, or elements.

As used herein, “at least one of” is synonymous with “one or more of”. For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

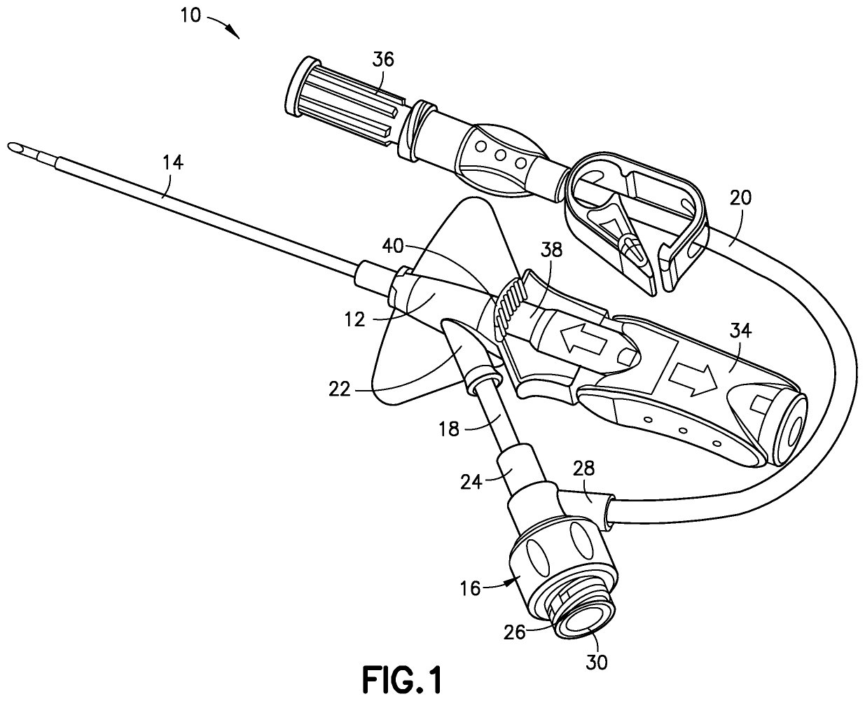

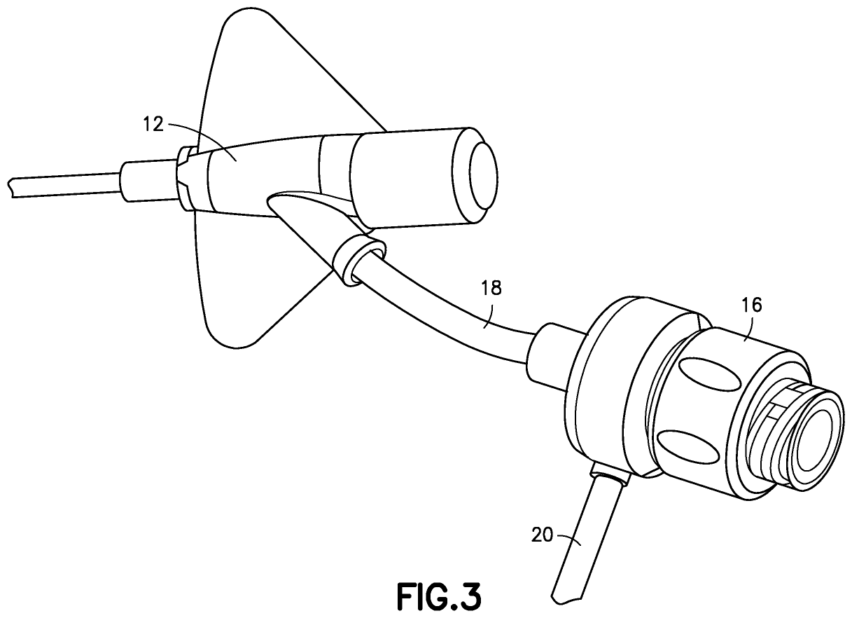

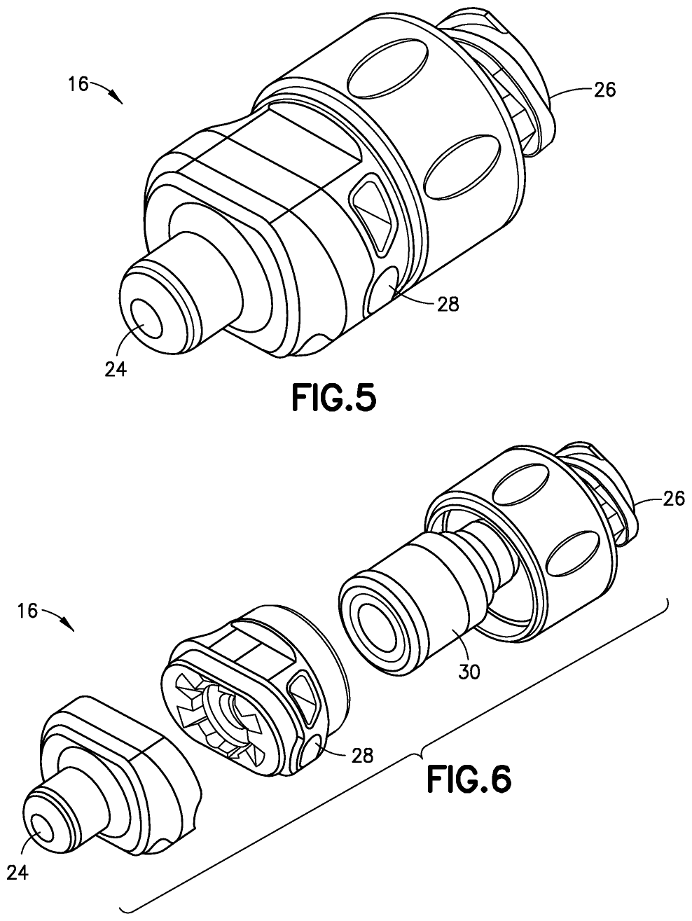

Referring to FIGS. 1-13, an integrated intravenous catheter 10 includes a catheter adapter 12 having a catheter 14 configured to be inserted into a patient's vasculature, a needle-free connector 16, intermediate tubing 18, and extension tubing 20. The catheter adapter 12 includes an inlet 22. The needle-free connector 16 includes a first port 24, a second port 26 positioned opposite the first port 24, and a side port 28 positioned between the first port 24 and the second port 26. The second port 26 includes a valve member 30. The intermediate tubing 18 extends between the inlet 22 of the catheter adapter 12 and the first port 24 of the needle-free connector 16. The extension tubing 20 extends from the side port 28 of the needle-free connector 16. The intermediate tubing 18 is configured to provide flexibility when inserting and dressing the catheter 14 and also when manipulating the needle-free connector 16 for flushing, blood draw, and/or other procedure without disturbing the catheter insertion site. The proximity of the needle-free connector 16 to the catheter adapter 12 is configured to enable a blood draw device 32 to protrude a sufficient distance beyond an end of the catheter 14. The extension tubing 20 is configured to be utilized for introducing of intravenous fluid, medication, etc.

In one aspect or embodiment, the intermediate tubing 18 has a length of 6-8 mm. In a further aspect or embodiment, the intermediate tubing 18 has a maximum length of 15 mm. A longer length of the intermediate tubing 18 improves insertion ergonomics, flexibility of access and securement, and ease of dressing. However, increasing the length of the intermediate tubing 18 decreases the distance the blood draw device 32 will be able to protrude from the catheter 14 unless the blood draw device 32 allows for a longer tubing. In one aspect or embodiment, the intermediate tubing 18 has a length that is shorter than a length of the extension tubing 20. The intermediate tubing 18 may have a length configured to allow a probe of the blood draw device 32 extend a predetermined length beyond a tip of the catheter 14.

Referring to FIG. 1, in one aspect or embodiment, the integrated catheter 10 includes a needle hub assembly 34 and a medical component 36, such as a vent plug. The needle hub assembly 34 is assembled with the catheter adapter 12 by inserting a needle (not shown) into a lumen of the catheter 14. In one aspect or embodiment, the needle hub assembly 34 includes a needle shield 38 configured to secure a tip of the needle within the needle shield 38 after use. The needle shield 38 may be activated passively. The needle hub assembly 34 includes a push tab 40 to facilitate catheter advancement during insertion. The push tab 40 also allows for one-handed or two-handed advancement. In one aspect or embodiment, the catheter adapter 12 includes one or more wings, as shown, configured to engage a skin surface of a patient. In another aspect or embodiment, the catheter adapter 12 does not include wings.

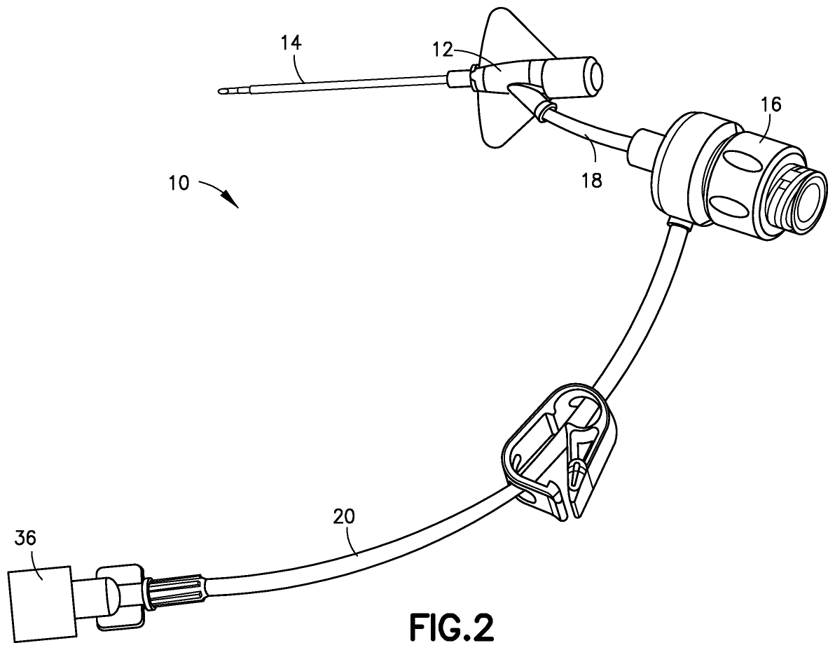

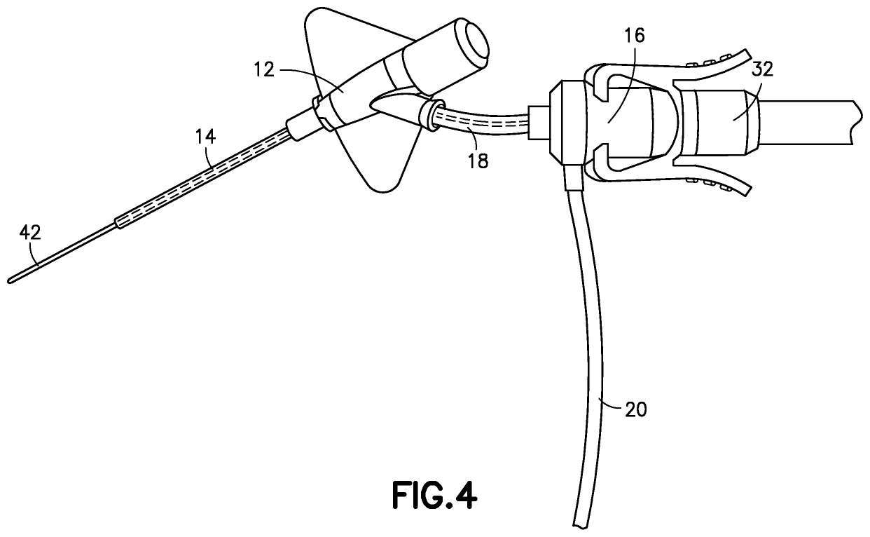

Referring to FIGS. 2-6, in one aspect or embodiment, the intermediate tubing 18 is fixedly connected to the inlet 22 of the catheter adapter 12 and the first port 24 of the needle-free connector 16. The intermediate tubing 18 is fixedly connected to the inlet 22 via bonding, adhesive, heat sealing, etc., although other suitable arrangements may be utilized. As shown in FIG. 4, when the blood draw device 32 is connected to the second port 26 of the needle-free connector 16, a flow tube 42 of the blood draw device 32 can be extended through the needle-free connector 16, through the intermediate tubing 18, the inlet 22 of the catheter adapter 12, and the catheter 14. The blood draw device 32 may be the PIVO™ blood draw device commercially available from Velano Vascular. In one aspect or embodiment, the blood draw device 32 is the same or similar to the blood draw device shown in U.S. Pat. No. 11,090,461, which is hereby incorporated by reference in its entirety. In one aspect or embodiment, the blood draw device 32 may be any device that advances tubing, a probe, a guidewire, instrument, and/or sensor into the fluid path of the integrated catheter 10 or beyond the tip of the catheter 14. In one aspect or embodiment, the needle-free connector 16 is similar and operates similarly to the connector shown and described in U.S. Patent Application Publication No. 2019/0160275, which is hereby incorporated by reference in its entirety.

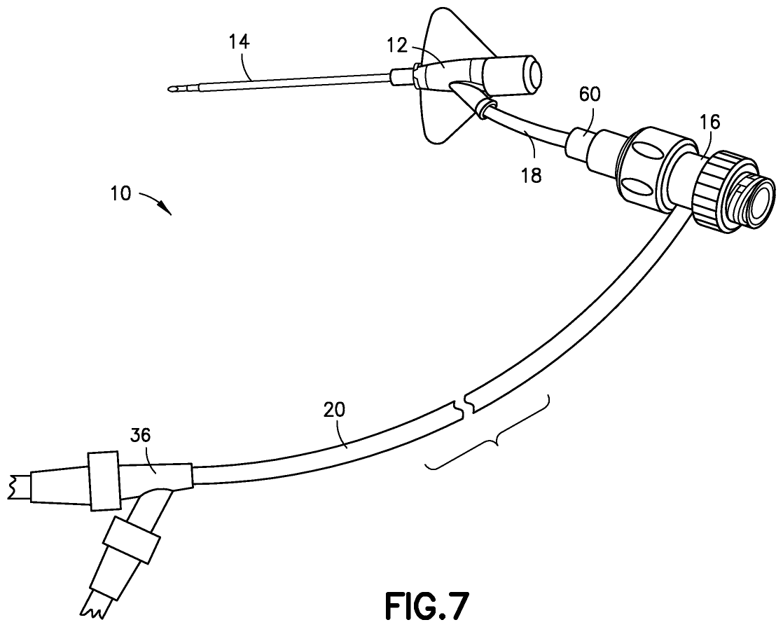

Referring to FIG. 7, in one aspect or embodiment, the first port 24 of the needle-free connector 16 is connected to the intermediate tubing 18 via a line connector 60, such as a luer connector. An end of the intermediate tubing 18 may include a male or female luer connector that is connected to a corresponding female or male luer connector provided at the first port 24 of the needle-free connector 16 to allow the needle-free connector 16 to be replaced without removing the catheter 14. As shown in FIG. 7, the medical component 36 is a dual port arrangement with two needle-free connectors.

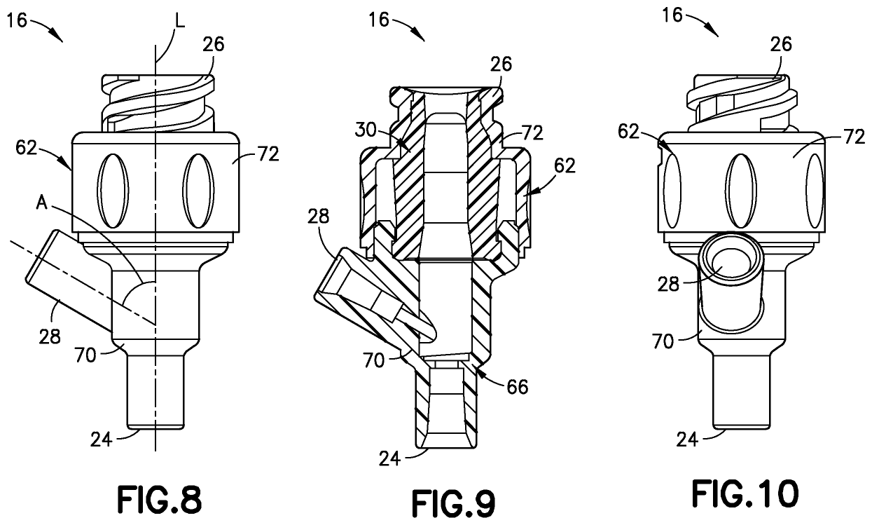

Referring to FIGS. 8-10, in one aspect or embodiment, the needle-free connector 16 includes a body 62 defining a flow path extending between the first port 24 and the second port 26. The side port 28 is offset from a center of the flow path. The offset of the side port 28 is configured to cause fluid entering the body 62 via the side port 28 to enter along an interior surface of the body 62 and cause a vortex within the body 62 to aid flushing of the needle-free connector 16. In one aspect or embodiment, the body 62 of the needle-free connector 16 further includes internal structure 66 configured to create a vortex when fluid enters the needle-free connector 16 via the side port 28. In one aspect or embodiment, the offset and vortex creating-feature is the same or similar to the flushing features shown and described in U.S. Patent Application Publication No. 2021/0220548, which is hereby incorporated by reference in its entirety.

Referring again to FIGS. 8-10, in one aspect or embodiment, the body 62 of the needle-free connector 16 defines a longitudinal axis L extending between the first port 24 and the second port 26, with the side port 28 extending from the body 62 at an angle A of 30-150 degrees relative to the longitudinal axis L of the body 62. In one aspect or embodiment, the side port 28 extends from the body 62 at an angle of 60 degrees relative to the longitudinal axis L of the body 62. The side port 28 extends at the angle A toward the second port 26, although other suitable arrangements may be utilized. The body 62 of the needle-free connector 16 includes a first portion 70 and a second portion 72 connected to the first portion 70. In one aspect or embodiment, the first portion 70 of the body 62 is fixedly secured to the second portion 72 of the body 62.

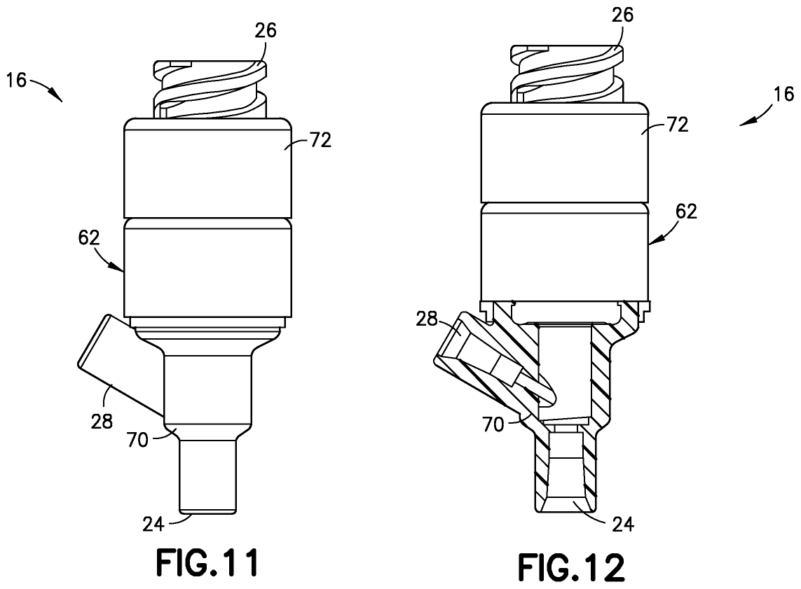

Referring to FIGS. 11 and 12, in one aspect or embodiment, the first portion 70 of the body 62 of the needle-free connector 16 is secured to the second portion 72 of the body 62 of the needle-free connector 16 via a luer connector. In one aspect or embodiment, the intermediate tubing 18 is connected to the first port 24 of the first portion 70 via a luer connection (not shown). The side port 28 may be provided on the first or second portions 70, 72 of the body 62 of the needle-free connector 16.

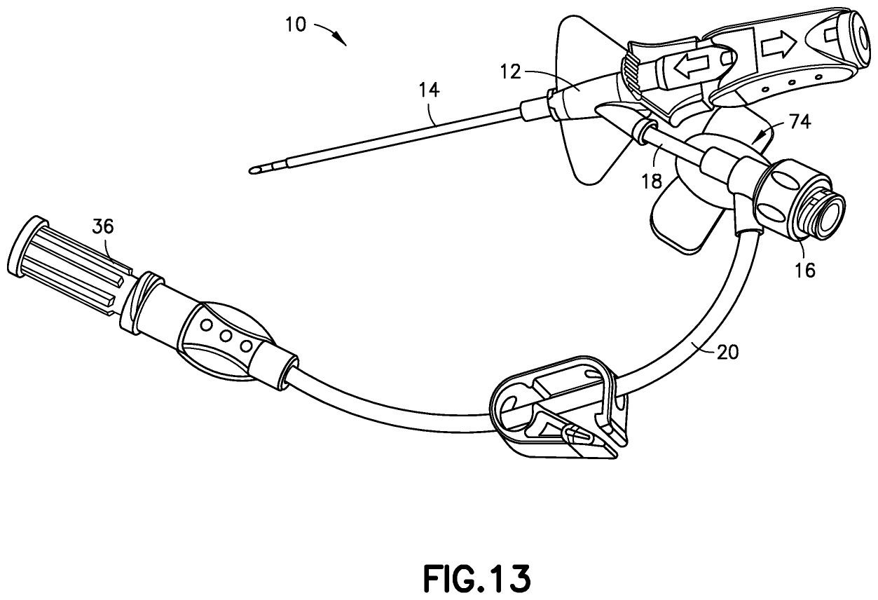

Referring to FIG. 13, in one aspect or embodiment, the needle-free connector 16 includes a stabilization member 74 configured to contact a skin surface of a patient during use of the integrated intravenous catheter 10. The stabilization member 74 may be formed integrally with the needle-free connector 16 or may be formed separately and connected to the needle-free connector 16 via clip or other securing arrangement. In one aspect or embodiment, the stabilization member 74 is overmolded onto the needle-free connector 16 and/or made from a softer material than the needle-free connector 16. In one aspect or embodiment, the stabilization member 74 is the same or similar to the stabilization member shown and described in U.S. Patent Application Publication No. 2019/0160275.

In one aspect or embodiment, at least a portion of the needle-free connector 16 is transparent. The connector components of the integrated catheter 10 may be transparent, opaque, and/or colored. In one aspect or embodiment, the needle-free connector 16 includes an anti-reflux valve.

In one aspect or embodiment, the medical component 36 at the end of the extension tubing 20 is a single port or dual port connector and may include a variety of connectors, including needle-free connectors or needle access connectors, such as a PRN. The extension tubing 20 may be left or right facing. In one aspect or embodiment, in addition to a vent plug, the medical component 36 may be a removable or non-removable needle free connector or needle access connectors, such as a PRN, that is attached to a female luer connection provided on the extension tubing 20. In one aspect or embodiment, a dual female luer port may be bonded or otherwise attached to the extension tubing 20 instead of a single luer connector.

In one aspect or embodiment, the needle-free connector 16 is configured to be flushed via a single flushing process via the side port 28.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

The invention claimed is:

1. A system comprising:
an integrated intravenous catheter comprising:
a catheter adapter comprising a catheter and an inlet, the catheter configured to be inserted into a patient's vasculature;
a needle-free connector comprising a first port, a second port positioned opposite the first port, and a side port positioned between the first port and the second port, the second port comprising a valve member;
intermediate tubing extending between the inlet of the catheter adapter and the first port of the needle-free connector; and
extension tubing extending from the side port of the needle-free connector; and
a blood draw device comprising a flow tube configured to be extended through the needle-free connector, through the intermediate tubing, through the inlet of the catheter adapter, and through the catheter.

2. The system of claim 1, wherein the needle-free connector comprises a body defining a flow path extending between the first port and the second port, and wherein the side port is offset from a center of the flow path.

3. The system of claim 1, wherein a length of the intermediate tubing is shorter than a length of the extension tubing.

4. The system of claim 1, wherein the intermediate tubing has a length configured to allow the flow tube of the blood draw device to extend a predetermined length beyond a tip of the catheter.

5. The system of claim 1, further comprising a medical connector positioned at an end of the extension tubing.

6. The system of claim 1, wherein the intermediate tubing is fixedly connected to the inlet of the catheter adapter and the first port of the needle-free connector.

7. The system of claim 1, wherein the first port of the needle-free connector is connected to the intermediate tubing via a line connector.

8. The system of claim 1, wherein the needle-free connector comprises a body defining a longitudinal axis extending between the first port and the second port, and wherein the side port extends from the body at an angle of 30-150 degrees relative to the longitudinal axis of the body.

9. The system of claim 8, wherein the body of the needle-free connector comprises a first portion and a second portion connected to the first portion via a luer connector.

10. The system of claim 1, wherein at least a portion of the needle-free connector is transparent.

11. The system of claim 1, wherein the needle-free connector comprises an anti-reflux valve.

12. The system of claim 1, wherein the needle-free connector comprises internal structure configured to create a vortex when fluid enters the needle-free connector via the side port.

13. The system of claim 1, wherein the needle-free connector comprises a stabilization member configured to contact a skin surface of a patient during use of the integrated intravenous catheter.

14. The system of claim 13, wherein the stabilization member is formed integrally with the needle-free connector.

15. The system of claim 14, wherein the stabilization member is separately connected to the needle-free connector.

16. A method of flushing the integrated catheter of claim 1, the method comprising:
disconnecting the blood draw device from the second port of the needle-free connector;
connecting a reservoir comprising a flushing fluid to the needle-free connector; and
flushing the first port, the second port, and the side port of the needle-free connector and the catheter of the catheter adapter with the flushing fluid via only one of the ports of the needle-free connector.

17. The method of claim 16, wherein the reservoir comprises a flush syringe or an intravenous fluid container.

18. The method of claim 16, wherein the reservoir is connected to the side port of the needle-free connector.
